(19) United States
(12) Patent Application Publication
(10) Pub. No.: US 2024/0194322 A1
(43) Pub. Date: Jun. 13, 2024

(54) SMART DESK FOR IDENTIFYING DECELERATION IN BRAIN ACTIVITY OF USER AND REMINDING THE USER TO RECOVER FROM THE SAME

(71) Applicant: Charcoal Technologies Inc., Wilmington, DE (US)

(72) Inventors: Arjun Ashok, Bengaluru (IN); Drishti Desai, Bengaluru (IN); Aniz Ali, Navi Mumbai (IN); Aswath S. Madhavan, Bangalore (IN); Supriya Nanjundegowada, Bangalore (IN); Karrthik G K, Bangalore (IN)

(21) Appl. No.: 18/530,108

(22) Filed: Dec. 5, 2023

(30) Foreign Application Priority Data
Dec. 7, 2022 (IN) 202241070722

(51) Int. Cl. G16H 20/70

(52) U.S. Cl. CPC G16H 20/70

(57) ABSTRACT

Disclosed is a system for identifying a deceleration in a brain activity of a user and reminding the user to take a break to recover from the deceleration of the brain activity. The system includes a smart desk including a sensor unit and a brain activity identification unit. The brain activity identification unit receives (i) user data (such as sleep data, wake up time, and age) from a user device on a particular day, and (ii) sensor data (for example, log-in time, and how long the user is performing the event) for the particular day from the sensor unit. The brain activity identification unit identifies the deceleration in the brain activity of the user based on the user data and the sensor data. The brain activity identification unit is further configured to remind the user to take the break to recover from the deceleration of the brain activity.

### BACKGROUND

### Technical Field

The embodiments herein generally relate to an interactive multi-functional furniture system, and more particularly, to a smart desk for identifying a deceleration in a brain activity of a user and reminding the user to take a break to recover from the deceleration of the brain activity.

### Description of the Related Art

In general, human brain works in a rhythm, alternating between peaks and slumps. More clearly, the neurons in the brain communicate by firing electrical impulses, which can be measured in terms of brain waves. The brain waves follow a cyclical pattern called an ultradian rhythm, where a brain activity increases steadily in the first part of the cycle (i.e., peak or acceleration in the brain activity) and then slows down considerably for a brief period (for example, about 20 minutes) (i.e., slump or a deceleration in the brain activity). Scientific reason behind this transition is that brain cells use sodium and potassium ions to fire the electrical impulses which get depleted during the acceleration in the brain activity, hence, the brain has to replenish them by going into a state of deceleration in the brain activity. The acceleration in the brain activity corresponds to states of alertness or focus of a person as the brain works at maximum capacity with full energy. The deceleration in the brain activity corresponds to fatigue, sleepiness, imagination, or daydreaming of the person, as the brain begins to crave a period of rest and recovery.

If the person works through the slumps during the deceleration in the brain activity without being aware of the natural rhythm, it is hard to prevent the brain from recovering properly, which leads to feeling worse during slurps and the lack of recovery energy for the next peak. That is, if the person forces a stressed brain to continue working during the slump, the brain pours out stress hormones such as adrenaline and cortisol to act as a substitute for the depleted sodium and potassium ions. When the brain relies on such hormones for energy, the stress builds up exponentially and the prefrontal cortex of the brain begins to shut down, resulting in less capability of thinking clearly. Further, the person is completely burnt out, if the stress hormones are depleted. If the person hit this completely ‘burnt-out state’, even taking a break becomes ineffective. Hence, it is very important to avoid such a state as it is difficult to reverse and ruins productivity completely.

There have been existed few reminders in the market, in a form of user devices, for example, watches and desks that remind the person to take a break in between performing an event (for instance, office work). For example, an Apple Watch that hourly reminds the person to stand. However, this watch is a cyclical reminder and is not synchronized with the person's actual slurps. Some existing desks remind the person about breaking timings based on data such as health data, sitting time on the desk, etc. However, these reminders are ineffective as they do not identify the slumps associated with the person and remind the person according to the identified slumps.

Therefore, there arises a need to address the aforementioned technical drawbacks in existing reminder systems in reminding the person to take a break while doing the event based on the ultradian rhythm including the slumps associated with the person.

### SUMMARY

In view of the foregoing, an embodiment herein provides a smart desk for identifying a deceleration in a brain activity of a user when the user performs an event at the smart desk and reminding the user to take a break to recover from the deceleration of the brain activity. The smart desk includes (a) a sensor unit that detects a presence of the user on the smart desk, a log-in time of the user at the smart desk, and how long the user is performing the event at the smart desk; and (b) a brain activity identification unit that includes a memory unit that stores a database. The database includes one or more energy graph templates, and each energy graph template includes one or more time periods at which a person has a deceleration in the brain activity. One or more time periods are pre-configured for each energy graph template based on the historical data of the person. In some embodiments, the historical data of the person includes at least one of the sleep data, a wakeup time, sensor data on the log-in time of the person on the smart desk, and how long the person performed the event at the smart desk, and an age of the person.

The brain activity identification unit further includes a microprocessor that is communicatively connected to the sensor unit and a user device associated with the user. The microprocessor is configured to (a) receive user data of a particular day from the user device wherein the user data includes at least one of sleep data, an age, and a wakeup time of the user on the particular day; (b) determine an energy graph template for the user for the particular day from the database by analyzing (i) the user data that is received from the user device, and (ii) the log-in time of the user on the smart desk that is received from the sensor unit, for the particular day; (c) determine an occupancy of the smart desk by the user on the particular day by analyzing information related to the presence of the user on the smart desk, and how long the user is performing the event at the smart desk; (d) dynamically modify the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user, for the particular day, wherein the adjusted energy graph includes one or more adjusted time periods on which the potential deceleration in the brain activity of the user occurs; (e) generate a reminder for each adjusted time period of the adjusted energy graph at a user interface on the smart desk to enable the user to take the break to recover from the deceleration of the brain activity.

In some embodiments, the sensor unit detects an infra-red radiation surrounding the smart desk to detect the presence of the user on the smart desk.

In some embodiments, the sensor unit includes a passive infra-red (PIR) sensor that detects the presence of the user on the smart desk, the log-in time of the user on the smart desk, and how long the user is performing the event at the smart desk.

In some embodiments, the user device includes a pre-determined application through which the user data is inputted by the user for the particular day. In some embodiments, the pre-determined application of the user device is paired with the smart desk via a network.

In some embodiments, the network is selected from a group comprising of Bluetooth, Long Range (LoRa), WiFi, Narrow Band-Internet of Things (NB-IoT), GSM/GPRS module, Zigbee WiFi, Radio Frequency (RF), Zigbee or combinations thereof.

In some embodiments, the data associated with the occupancy of the smart desk by the user includes a time duration for which the user occupies the smart desk for performing the event and a time duration on which the user takes rest.

In some embodiments, the user interface includes a Light Emitting Diode (LED) that visually indicates the deceleration in the brain activity of the user by changing colour.

In some embodiments, the deceleration of the brain activity of the user includes a state attained by the user including at least one of fatigue, slump, sleepiness, imagination, daydreaming, irritable feeling, stressed, or lack of drive and motivation.

In one aspect, a method of identifying a deceleration in a brain activity of a user when the user performs an event at the smart desk and reminding the user to take a break to recover from the deceleration of the brain activity is provided. The method includes (a) providing the smart desk that includes a sensor unit, and a brain activity identification unit including a memory unit, and a microprocessor; (b) pre-storing one or more energy graph templates on a database in the memory unit, each energy graph template includes one or more time periods at which a person has the deceleration in the brain activity, wherein the one or more time periods are pre-configured for each energy graph template based on historical data of the person, wherein the historical data of the person includes at least one sleep data, a wakeup time, sensor data on log-in time of the person on the smart desk, and how long the person performed the event at the smart desk, and an age of the person; (c) detecting, using the sensor unit, a presence of the user on the smart desk, a log-in time of the user at the smart desk, and how long the user is performing the event at the smart desk; (d) receiving, by the microprocessor, user data of a particular day from a user device associated with the user, wherein the user data includes at least one of sleep data, age, and a wakeup time of the user for the particular day; (e) determining, by the microprocessor, an energy graph template for the user for the particular day from the database by analyzing (i) the user data that is received from the user device, and (ii) the log-in time of the user on the smart desk that is received from the sensor unit, for the particular day; (f) determining, by the microprocessor, an occupancy of the smart desk by the user on the particular day by analyzing information related to the presence of the user on the smart desk, and how long the user is performing the event at the smart desk; (g) dynamically modifying, by the microprocessor, the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user for the particular day, wherein the adjusted energy graph includes one or more adjusted time periods on which the potential deceleration in the brain activity of the user occurs; (h) generating, by the microprocessor, a reminder for each adjusted time period of the adjusted energy graph at a user interface on the smart desk to enable the user to take the break to recover from the deceleration of the brain activity.

In some embodiments, the method includes detecting, by the sensor unit, an infra-red radiation surrounding the smart desk to detect the presence of the user on the smart desk.

In some embodiments, the sensor unit includes a passive infra-red (PIR) sensor that detects the presence of the user on the smart desk, the log-in time of the user on the smart desk, and how long the user is performing the event at the smart desk.

In some embodiments, the method includes inputting, by the user, the user data to the user device for the particular day through a pre-determined application.

In some embodiments, the method includes pairing the pre-determined application of the user device with the smart desk via a network.

In some embodiments, the network is selected from a group comprising of Bluetooth, Long Range (LoRa), WiFi, Narrow Band-Internet of Things (NB-IoT), GSM/GPRS module, Zigbee WiFi, Radio Frequency (RF), Zigbee or combinations thereof.

In some embodiments, the data associated with the occupancy of the smart desk by the user comprises a time duration for which the user occupies the smart desk for performing the event, and a time duration for which the user takes rest

In some embodiments, the method includes indicating, by the microprocessor, the deceleration of the brain activity of the user by changing colour of the user interface that includes a Light Emitting Diode (LED).

In some embodiments, the deceleration of the brain activity of the user comprises a state attained by the user comprising at least one of fatigue, slump, sleepiness, imagination, daydreaming, irritable feeling, stressed, or lack of drive and motivation.

In another aspect, one or more non-transitory computer readable storage mediums storing one or more sequences of instructions, which when executed by one or more processors, performs a method of identifying a deceleration in a brain activity of a user when the user performs an event at the smart desk and reminding the user to take a break to recover from the deceleration of the brain activity is provided. The method includes (a) providing the smart desk that includes a sensor unit, and a brain activity identification unit including a memory unit, and a microprocessor; (b) pre-storing one or more energy graph templates on a database in the memory unit, each energy graph template includes one or more time periods at which a person has the deceleration in the brain activity, wherein the one or more time periods are pre-configured for each energy graph template based on historical data of the person, wherein the historical data of the person includes at least one sleep data, a wakeup time, sensor data on log-in time of the person on the smart desk, and how long the person performed the event at the smart desk, and an age of the person; (c) detecting, using the sensor unit, a presence of the user on the smart desk, a log-in time of the user at the smart desk, and how long the user is performing the event at the smart desk; (d) receiving, by the microprocessor, user data of a particular day from a user device associated with the user, wherein the user data includes at least one of sleep data, age, and a wakeup time of the user for the particular day; (e) determining, by the microprocessor, an energy graph template for the user for the particular day from the database by analyzing (i) the user data that is received from the user device, and (ii) the log-in time of the user on the smart desk that is received from the sensor unit, for the particular day; (f) determining, by the microprocessor, an occupancy of the smart desk by the user on the particular day by analyzing information related to the presence of the user on the smart desk, and how long the user is performing the event at the smart desk; (g) dynamically modifying, by the microprocessor, the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user for the particular day, wherein the adjusted energy graph includes one or more adjusted time periods on which the potential deceleration in the brain activity of the user occurs; (h) generating, by the microprocessor, a reminder for each adjusted time period of the adjusted energy graph at a user interface on the smart desk to enable the user to take the break to recover from the deceleration of the brain activity.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a block diagram of a system for identifying a deceleration in a brain activity of a user and reminding the user to take a break to recover from the deceleration of the brain activity according to some embodiments herein;

FIG. 2 is a block diagram of a smart desk of FIG. 1 for identifying a deceleration in a brain activity of a user and reminding the user to take a break to recover from the deceleration of the brain activity according to some embodiments herein;

FIG. 3 is an exploded view of a brain activity identification unit of FIG. 2 according to some embodiments herein;

FIG. 4A is an exemplary view of an energy graph template that is pre-stored in a database of FIG. 3 according to some embodiments herein;

FIG. 4B is an exemplary view of an adjusted energy graph that is obtained by modifying the energy graph template of FIG. 4A according to some embodiments herein;

FIGS. 5A-5C are exemplary interface views of a user device of FIG. 1 according to some embodiments herein;

FIGS. 6A and 6B are flow diagrams that illustrate a method of identifying a deceleration in a brain activity of a user and reminding the user to take a break to recover from the deceleration of the brain activity using a smart desk of FIG. 2 according to some embodiments herein; and

FIG. 7 is a schematic diagram of computer architecture of a computing device, in accordance with the embodiments herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

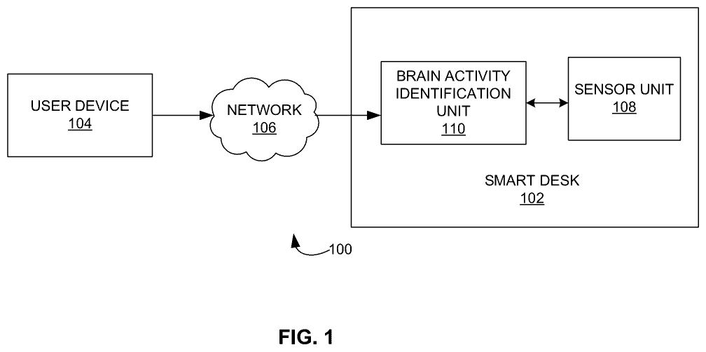

As mentioned, there remains a need for a reminder system to remind a person to take a break while doing an event based on an ultradian rhythm associated with the person. The embodiments herein achieve this by proposing a smart desk that identifies a deceleration in a brain activity of a user when the user performs an event at the smart desk and reminds the user to take a break to recover from the deceleration of the brain activity. Referring now to the drawings, and more particularly to FIGS. 1 through 7, where similar reference characters denote corresponding features consistently throughout the figures, there are shown preferred embodiments.

As used herein, several terms are defined below:
The term “smart desk” used herein refers to any furniture with surface areas which uses intelligent technology to help people be healthier and more productive;
The term “ultradian rhythm” used herein refers to repeated biological patterns, especially psychological patterns that have a cycle of less than 24 hours, where a brain activity increases steadily in a first part of the cycle as a brain of a person works at maximum capacity with full energy and then slows down considerably for a brief period as the brain begins to crave a period of rest and recovery;
The term “brain activity” used herein refers to an ability of the person to perform an event;
The term “acceleration in a brain activity” used herein refers to a state of alertness or focus of the person on the event, at which the person can be able to perform the event effectively with full energy;
The term “deceleration in a brain activity” or “ultradian slump” used herein refers to a state of at least one of fatigue, slump, sleepiness, imagination, daydreaming, stressed, irritable feeling of the person, at which the person cannot perform the event effectively;
The term “event” used herein refers to any cognitive or physiological task of the person including office work, writing work, reading work, studying, or any activity performed at the smart desk;
The terms “energy graph template” and “adjusted energy graph” used herein refer to a time data source which includes one or more time periods that dictates at what times of the day, the person has a deceleration in the brain activity. The energy graph template has predefined time periods and the adjusted energy graph has adjusted time periods;
The term “one or more time periods” used herein refers to time durations having time intervals in between. Time intervals on which the user has the acceleration in the brain activity, hence performs the event. Time durations at which the user has the deceleration in the brain activity, hence the user is reminded to take a break.
The term “one or more adjusted time periods” used herein refers to time durations having modified time intervals in between.
The term “occupancy of the smart desk” used herein refers to residing of the person on the smart desk for performing the event;
The term “pre-determined application” used herein refers to a mobile or web application that is specifically created for smart desk usage applications;
The term “log-in time” refers to when the person starts to perform the event on the smart desk 102 for the first time on the particular day; and
The term “take a break” used herein refers to an act of stop performing the event for a short period of time.

FIG. 1 is a block diagram of a system 100 for identifying a deceleration in a brain activity of a user and reminding the user to take a break to recover from the deceleration of the brain activity according to some embodiments herein. The system 100 includes a smart desk 102, a user device 104, and a network 106. The smart desk 102 includes a working surface that provides a space for the user to perform an event at the smart desk 102. The event may be any task of the user including office work, writing work, reading work, studying, or any activity performed at the smart desk 102. The smart desk 102 further includes a sensor unit 108, and a brain activity identification unit 110.

The brain activity identification unit 110 is communicatively connected with the user device 104 associated with the user through the network 106 to receive the user data from the user device 104. The user data includes at least one of sleep data, an age, and a wakeup time of the user on a particular day. In some embodiments, the user device 104 obtains the sleep data and the wake up time with one or more in-built sleep monitoring sensors associated with one or more programs that are installed in the user device 104. The one or more programs may be a client-side application. In some embodiments, the wake up time and the age of the user are manually entered into the user device 104 by the user. The sleep data and the wake up time may be obtained by the user using one or more sleep monitoring sensors that are integrated in at least one of bed, pillow, or any wearable of the user. The user device 104 may obtain the sleep data and the wake up time by communicating with the one or more sleep monitoring sensors. The one or more sleep monitoring sensors may include a three-axis accelerometer, a pressure sensor, a piezo film sensor, a respiration sensor, a heartbeat sensor, or any kind of sleep monitoring sensors.

The user device 104 may include a predetermined application through which the user data is inputted by the user for the particular day. The predetermined application may be configured to obtain sleep data of the user. The predetermined application may be a mobile application or a web application. The predetermined application of the user device 104 may be paired with the smart desk 102 through the network 106 for transmitting the user data to the smart desk 102. The user device 104 may be a handheld device, a mobile phone, a kindle, a Personal Digital Assistant (PDA), a tablet, a music player, a computer, an electronic notebook or a Smartphone. The network 106 may be selected from a group consisting of Bluetooth, Long Range (LoRa), WiFi, Narrow Band-Internet of Things (NB-IoT), GSM/GPRS module, Zigbee WiFi, Radio Frequency (RF), Zigbee or combinations thereof.

The brain activity identification unit 110 is further configured to receive the sensor data for the particular day from the sensor unit 108 through a communication network. The sensor unit 108 is configured to detect a presence of the user on the smart desk 102 and obtains the sensor data with a timestamp. The sensor data may include information on (a) when the user starts the event at the smart desk 102, that is, a log-in time of the user on the smart desk 102 on the particular day; and (b) how long the user is performing the event at the smart desk 102. The communication network may be a wireless network or wired network.

The brain activity identification unit 110 identifies the deceleration in the brain activity of the user when the user performs the event at the smart desk 102 based on the user data and the sensor data. The deceleration of the brain activity of the user includes a state attained by the user including at least one of fatigue, slump, sleepiness, imagination, daydreaming, irritable feeling, stressed, or lack of drive and motivation. The brain activity identification unit 110 is further configured to remind the user to take the break to recover from the deceleration of the brain activity.

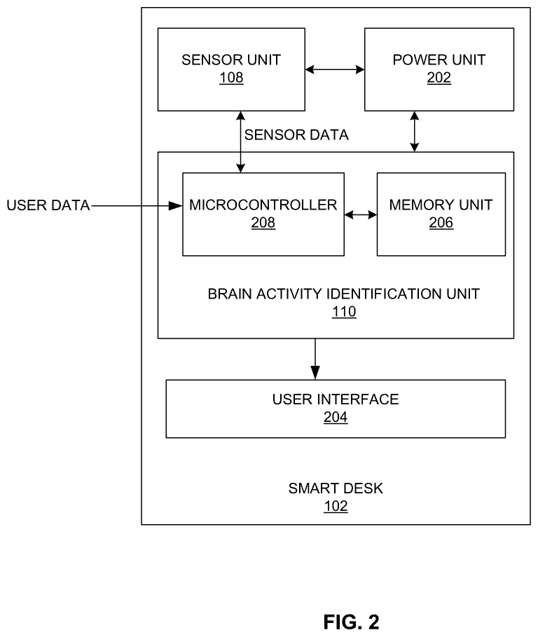

FIG. 2 is a block diagram of a smart desk 102 of FIG. 1 for identifying a deceleration in a brain activity of a user and reminding the user to take a break to recover from the deceleration of the brain activity according to some embodiments herein. The smart desk 102 includes a power unit 202, the sensor unit 108, the brain activity identification unit 110, and a user interface 204.

The power unit 202 energizes the smart desk 102 for its operation. The smart desk 102 may receive energy from a power supply unit directly or stores the energy from the power supply unit in the power storage unit for the operation of the smart desk 102. The power supply unit may be a grid, a power bank or any external power supply means. The power storage unit may be a battery. The smart desk 102 may be portable from one place to another. The smart desk 102 may be rechargeable.

The brain activity identification unit 110 includes a memory unit 206 and a microprocessor 208. The memory unit 206 stores a database and a set of modules of the brain activity identification unit 110. The microprocessor 208 executes the set of modules in the memory unit 206 for identifying the deceleration in the brain activity of the user.

The memory unit 206 pre-stores one or more energy graph templates on the database. Each energy graph template includes one or more time periods at which a person has the deceleration in the brain activity. The one or more time periods are pre-configured for each energy graph template based on historical data of the person. The historical data of the person includes at least one sleep data, a wakeup time, sensor data on log-in time of the person on the smart desk 102 and how long the person performed the event at the smart desk 102, and an age of the person.

The user starts to perform the event at the smart desk 102 after logging into the smart desk 102 using the user device 104 through the predetermined application using a login method with login information. The login method may be at least one of a password-based authentication with the login information, for example, user name/password, a biometric authentication including face recognition, fingerprint identification, voice recognition, and eye scanning, or a gesture-based authentication. One or more users may use the smart desk 102 at different times. The smart desk 102 may allow a new user only after logging in through the predetermined application associated with the user device 104 with the login information. In some embodiments, the smart desk 102 may include a login interface that allows the user to login to the smart desk 102.

When the user logs in to the smart desk 102 using the user device 104 through the predetermined application for the first time, the microprocessor 208 receives the login information from the user device 104 through the network 106 and verifies the login information with a stored identity of the user. The identity of the user may be a password, a voice, fingerprints, eye patterns, face of the user, or specific gestures, and may be pre-stored in the database. When the user device 104 disconnects from the smart desk 102 by going away from the proximity of the smart desk 102 (for example, nearly 30 feet or 10 meters) and comes within the proximity of the smart desk 102 again, the smart desk 102 automatically verifies the login information of the user device 104. Further, the smart desk 102 automatically connects the user device 104 an enable the user to perform the event at the smart desk 102, if the user device 104 is already logged into the smart desk 102.

The sensor unit 108 is configured to detect a presence of the user on the smart desk 102 and obtains the sensor data with a timestamp. For example, if the sensor unit 108 detects the presence of the user at 10:31:32, then the timestamp data associated with the presence detection is 10:31:32. The sensor data may include (a) when the user starts the event at the smart desk 102, that is, a log-in time of the user on the smart desk 102; and how long the user is performing the event at the smart desk 102. The sensor unit 108 may detect the presence of the user at every pre-determined length of time or when the user approaches the smart desk 102. The sensor unit 108 may be at the rest until the presence of user is detected.

In some embodiments, the sensor unit 108 detects an infrared radiation surrounding the smart desk 102 to detect the presence of the user on the smart desk 102. The infrared radiation may be emitted by the user as a result of temperature. The sensor unit 108 may be a passive infra-red (PIR) sensor that obtains the sensor data with the timestamp data. The sensor unit 108 may be an active infra-red sensor, a radar-based human sensor, a laser-based sensor, a microwave based sensor, a motion detector, an acoustic sensor, and the like. In some embodiments, the sensor unit 108 is integrated at any place at the smart desk 102. The sensor unit 108 may be placed at any place near the smart desk 102 and may be communicatively connected with the smart desk 102 using the communication network.

The microprocessor 208 receives user data from the user device 104 associated with the user for a particular day through the network 106. The user data includes at least one of sleep data, an age, and a wakeup time of the user on the particular day. The microprocessor 208 further receives the sensor data for the particular day from the sensor unit 108 through the communication network. The user data and the sensor data may be stored in the database. The particular day may refer to a day on which the user starts the event at the smart desk 102.

The microprocessor 208 is further configured to select a suitable energy graph template for the user for the particular day from the database by analyzing (i) the user data that is received from the user device 104, and (ii) the log-in time of the user on the smart desk 102 that is received from the sensor unit 108, for the particular day. For example, if the age of the user is 38, the wake up time of the user is 6.00 am, number of hours slept last night (i.e., sleep data) is 7 hours, and the log-in time of the user is 10.00 am, the microprocessor 208 selects, for example, a first energy graph template that is pre-configured with time periods having time duration of two hour interval from the login-in time of the user, i.e. the time periods may be 12.00-12.15 pm; 2.15-2.30 pm; 4.30-4.45 pm; and the like. If the age of the user is 30, the wake up time of the user is 6.30 am, number of hours slept last night (i.e., sleep data) is 5 hours, and the log-in time of the user is 9.30 am, the microprocessor 208 selects, for example, a second energy graph template that is pre-configured with time periods having time duration of half-an-hour interval from the login-in time of the user, i.e. the time periods may be 10.00-10.15 am; 10.45-11.00 am; 11.30-11.45 am; and the like. One energy graph template may be selected for one user per day based on user data and sensor data. The energy graph template may be different for different users.

The microprocessor 208 further determines occupancy of the smart desk 102 by the user on the particular day by analyzing information related to the presence of the user on the smart desk 102 and how long the user is performing the event at the smart desk 102. The occupancy of the smart desk 102 may be determined at regular interval of time. The microprocessor 208 dynamically modifies the energy graph template of the user for the particular day by adjusting the one or more time periods of the energy graph template to obtain an adjusted energy graph for the user. The one or more time periods of the energy graph template are adjusted by analyzing data associated with the occupancy of the smart desk 102 by the user. The data associated with the occupancy of the smart desk 102 includes a time duration for which the user occupies the smart desk 102 for performing the event and a time duration on which the user takes rest. Whenever the data associated with the occupancy of the smart desk 102 is determined, the one or more time periods of the energy graph template are adjusted, and the adjusted energy graph is obtained each time for the user. The adjusted energy graph includes one or more adjusted time periods on which the potential deceleration in the brain activity of the user occurs. For example, if the first energy graph template is selected for the user, and the user has occupied the smart desk 102 for 3 hours continuously from 10.00 am to 1.00 pm for performing the event, the microprocessor 208 adjusts the time periods of the first energy graph template to have the time duration of one hour interval from the two hours of interval (i.e., actual time duration, the first energy graph template has) and provides a first adjusted energy graph for the user with the adjusted time periods having the time duration of one hour interval.

The microprocessor 208 is further configured to generate a reminder for each adjusted time period of the adjusted energy graph at the user interface 204 on the smart desk 102. The smart desk 102 may detect the presence of the user using the sensor unit 108 before notifying the user through the reminder. The reminder may be in at least one form of an alarm, a visual indication, an audio output, a vibration, or any suitable notification means. In some embodiments, the user interface 204 includes a Light Emitting Diode (LED) that visually indicates the user whether the user attains the deceleration in the brain activity by changing the colour of the LED from one to another. For example, the microprocessor 208 indicates the deceleration in the brain activity of the user by controlling the user interface 204 (LED) to visualize in green color on the adjusted time period. Before and after the adjusted time period, the LED may be in white color. The microprocessor 208 may visually indicate the user by turning ON or OFF the LED.

In some embodiments, the reminder may be generated at the user device 104 associated with the user. The reminder at the user device 104 may be in at least one form of an alarm, a visual indication, audio output, vibration, a phone call, a Short Message Service (SMS), or any suitable notification means.

Thus, the smart desk 102 enables the user to take the break to recover from the deceleration of the brain activity.

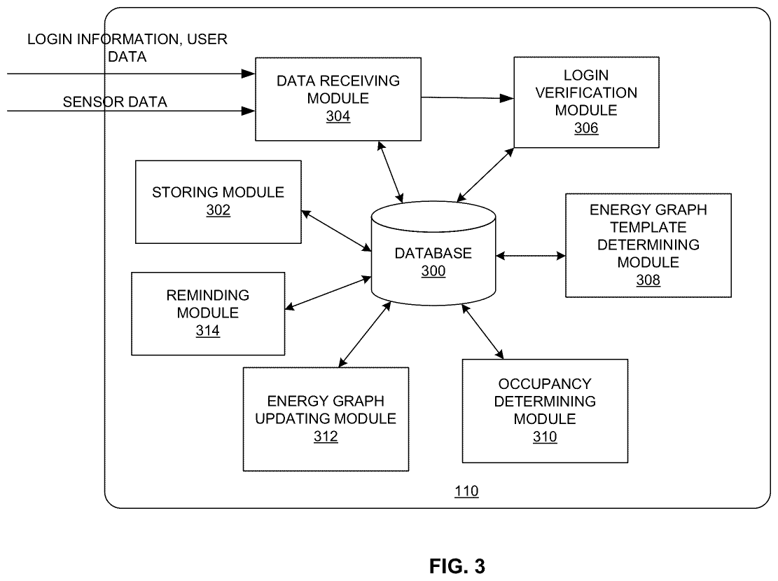

FIG. 3 is an exploded view of a brain activity identification unit 110 of FIG. 2 according to some embodiments herein. The brain activity identification unit 110 includes a database 300, a storing module 302, a data receiving module 304, a login verification module 306, an energy graph template determining module 308, an occupancy determining module 310, an energy graph updating module 312, and a reminding module 314.

The storing module 302 pre-stores one or more energy graph templates on the database 300. Each energy graph template includes one or more time periods at which a person has the deceleration in the brain activity. The data receiving module 304 receives login information from the user through the user device 104 associated with the user via the network 106.

The login verification module 306 verifies the login information with a user identity that is stored in the database 300 for the first time. The user logins into the smart desk 102 if the login is successful. When the user device 104 disconnects from the smart desk 102 and comes within the proximity of the smart desk 102 again, the login verification module 306 automatically verifies the login information of the user and automatically connects the user device 104 to enable the user to perform the event at the smart desk 102. When the user approaches the smart desk 102 to perform the event, the sensor unit 108 initiates a presence detection of the user at the smart desk 102 to obtain sensor data with a timestamp.

The data receiving module 304 further receives (i) user data from the user device 104 through the network 106, and (ii) the sensor data from the sensor unit 108 through the communication network for a particular day. The user data includes at least one of sleep data, an age, and a wakeup time of the user on the particular day. The sensor data may include (a) when the user starts the event at the smart desk 102, that is, a log-in time of the user on the smart desk 102 on the particular day and (b) how long the user is performing the event at the smart desk 102.

The energy graph template determining module 308 selects a suitable energy graph template for the user for the particular day from the database 300 by analyzing (i) the user data that is received from the user device 104, and (ii) the log-in time of the user on the smart desk 102 that is received from the sensor unit 108, for the particular day.

The occupancy determining module 310 determines occupancy of the smart desk 102 by the user on the particular day by analyzing information related to the presence of the user on the smart desk 102 and how long the user is performing the event at the smart desk 102.

The energy graph updating module 312 dynamically modifies the energy graph template of the user for the particular day by adjusting the one or more time periods of the energy graph template to obtain an adjusted energy graph for the user. The one or more time periods of the energy graph template are adjusted by analyzing data associated with the occupancy of the smart desk 102 by the user. The data associated with the occupancy of the smart desk 102 includes a time duration for which the user occupies the smart desk 102 for performing the event and a time duration on which the user takes rest. The adjusted energy graph includes one or more adjusted time periods on which the potential deceleration in the brain activity of the user occurs.

The reminding module 314 generates a reminder for each adjusted time period of the adjusted energy graph at the user interface 204 on the smart desk 102. The reminder may be in at least one form of an alarm, a visual indicator, audio output, vibrator, or any suitable notification means. The user interface 204 may include a Light Emitting Diode (LED) that visually indicates the user whether the user attains the deceleration in the brain activity by changing the colour of the LED from one to another.

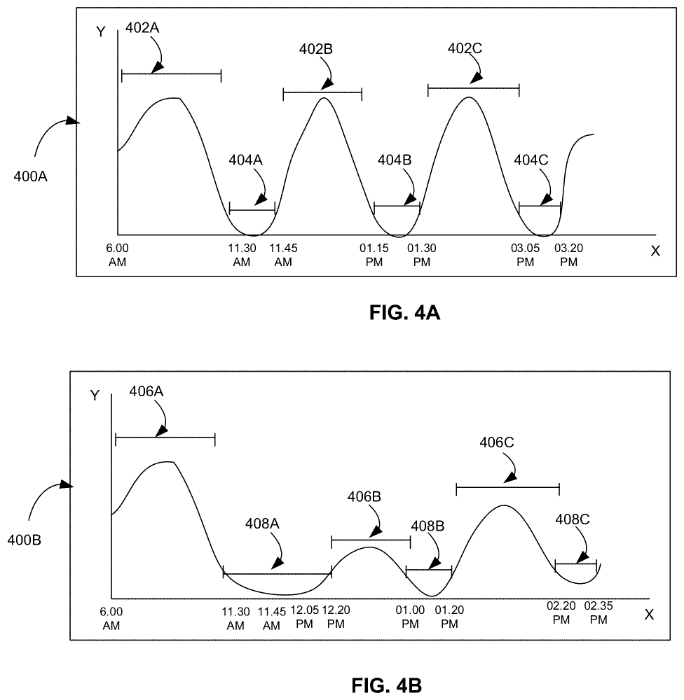

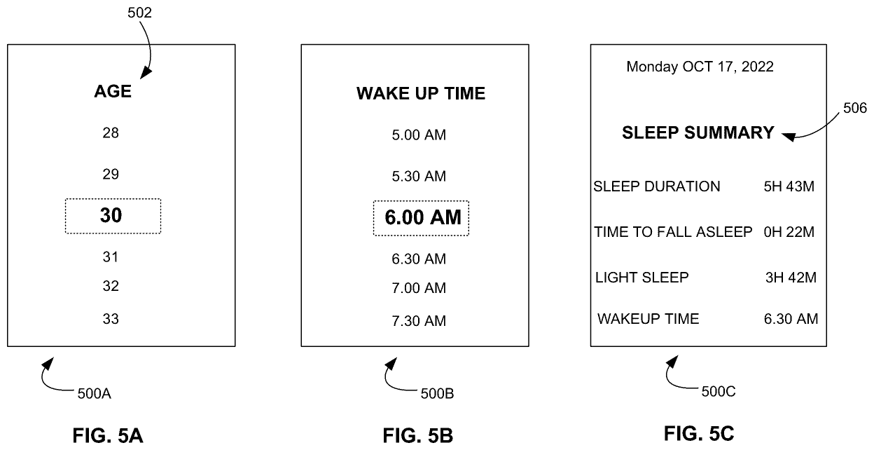

FIG. 4A is an exemplary view of an energy graph template 400A that is pre-stored in a database 300 of FIG. 3 according to some embodiments herein. The energy graph template 400A depicts one or more time periods that are plotted in an X-axis, and a brain activity of a user associated with the one or more time periods are plotted in a Y-axis. The energy graph template 400A may be selected based on user data, and a log-in time of the user on the smart desk 102. The peaks 402A, 402B, and 402C depict time intervals such as 6.00 am to 11.30 am; 11.45 am to 1.15 pm; and 1.30 pm to 3.05 pm at which the user has acceleration in the brain activity. The troughs 404A, 404B, and 404C depict time durations such as 11.30 am to 11.45 am; 1.15 pm to 1.30 pm; and 3.05 pm to 3.20 pm at which the user has a deceleration in the brain activity.

FIG. 4B is an exemplary view of an adjusted energy graph 400B that is obtained by modifying the energy graph template 400A of FIG. 4A according to some embodiments herein. The adjusted energy graph 400B depicts one or more adjusted time periods that are plotted in an X-axis, and a brain activity of a user associated with the one or more adjusted time periods are plotted in a Y-axis. The one or more time periods of the energy graph template 400A are adjusted by analyzing data associated with the occupancy of the smart desk 102 by the user. The peaks 406A, 406B, and 406C depict time intervals such as 6.00 am to 11.30 am; 12.20 am to 1.00 pm; and 1.20 pm to 2.20 pm at which the user has acceleration in the brain activity. The troughs 408A, 408B, and 408C depict time durations such as 11.30 am to 12.20 pm; 1.00 pm to 1.20 pm; and 2.20 pm to 2.35 pm at which the user has a deceleration in the brain activity which are reminded to the user through a user interface.

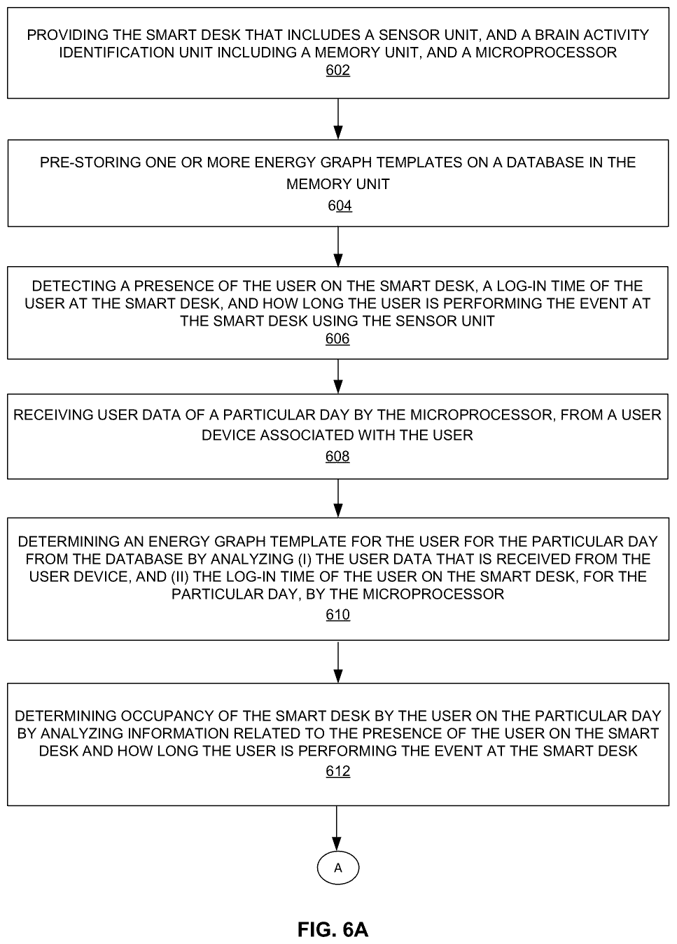

FIGS. 5A-5C are exemplary interface views of a user device 104 of FIG. 1 according to some embodiments herein. The user device 104 includes a predetermined application through which user data is inputted by the user. As shown in FIG. 5A, the interface view 500A of the predetermined application includes an age selecting interface 502 through which an age of the user is inputted by the user. As shown in FIG. 5B, the interface view 500B of the predetermined application includes a wake up time selection interface 504, through which a wakeup time of the user is inputted by the user. The user device 104 may include a program (for example, a sleep tracking mobile application) that obtains sleep data of the user. As shown in FIG. 5C, the interface view 500C of the program includes the sleep data 506 of the user. The sleep data may be communicated to the pre-determined application or manually inputted by the user.

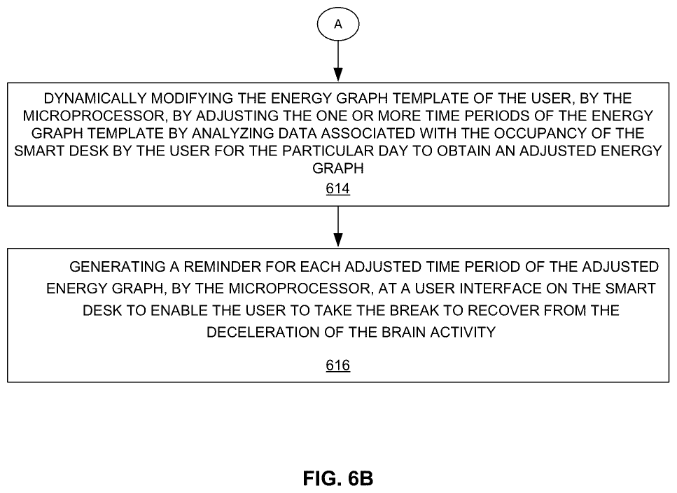

FIGS. 6A and 6B are flow diagrams that illustrate a method of identifying a deceleration in a brain activity of a user and reminding the user to take a break to recover from the deceleration of the brain activity using a smart desk 102 of FIG. 2 according to some embodiments herein. At step 602, the smart desk 102 is provided. The smart desk 102 includes a sensor unit 108, and a brain activity identification unit 110 including a memory unit 206, a microprocessor 208. At step 604, one or more energy graph templates are pre-stored on a database in the memory unit 206. Each energy graph template includes one or more time periods at which a person has the deceleration in the brain activity.

At step 606, a presence of the user on the smart desk 102, a log-in time of the user at the smart desk 102, and how long the user is performing the event at the smart desk 102 are detected using the sensor unit 108. At step 608, user data of a particular day is received, by the microprocessor 208, from a user device 104 associated with the user. The user data includes at least one of sleep data, age, and a wakeup time of the user for the particular day.

At step 610, an energy graph template for the user for the particular day is determined from the database by analyzing (i) the user data that is received from the user device 104, and (ii) the log-in time of the user on the smart desk 102, for the particular day, by the microprocessor 208. At step 612, occupancy of the smart desk 102 by the user on the particular day is determined by analyzing information related to the presence of the user on the smart desk 102 and how long the user is performing the event at the smart desk 102.

At step 614, the energy graph template of the user is dynamically modified, by the microprocessor 208, by adjusting the one or more time periods of the energy graph template by analyzing data associated with the occupancy of the smart desk 102 by the user for the particular day to obtain an adjusted energy graph. The adjusted energy graph includes one or more adjusted time periods on which the potential deceleration in the brain activity of the user occurs.

At step 616, a reminder for each adjusted time period of the adjusted energy graph is generated, by the microprocessor 208, at a user interface 204 on the smart desk 102 to enable the user to take the break to recover from the deceleration of the brain activity.

As the one or more time periods in the one or more energy graph templates are calibrated based on ultradian rhythm of human brain using sensor data and user data of the person, the smart desk 102 helps the user to identify when the user hit ultradian slumps and reminds the user to recover from the slumps. A quick break during the time periods of slumps helps the user to feel less tired, and to recover the energy for the next peak, which in turn improves productivity of the event.

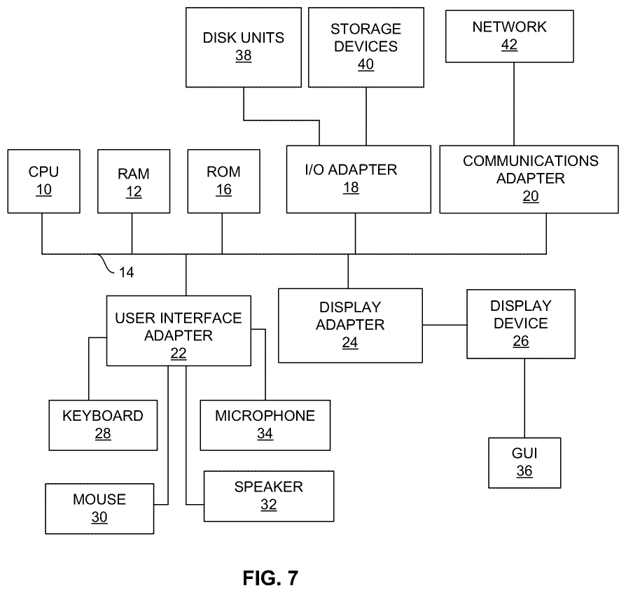

A representative hardware environment for practicing the embodiments herein is depicted in FIG. 7, with reference to FIGS. 1 through 6B. This schematic drawing illustrates a hardware configuration of a server/computer system/computing device in accordance with the embodiments herein. The system includes at least one processing device CPU 10 that may be interconnected via system bus 14 to various devices such as a random access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A smart desk for identifying a deceleration in a brain activity of a user when the user performs an event at the smart desk and reminding the user to take a break to recover from the deceleration of the brain activity, wherein the smart desk comprising,
a sensor unit that detects a presence of the user on the smart desk, a log-in time of the user at the smart desk, and how long the user is performing the event at the smart desk; and
a brain activity identification unit that comprises,
a memory unit that stores a database, wherein the database comprises a plurality of energy graph templates, and each energy graph template comprises one or more time periods at which a person has the deceleration in the brain activity, wherein the one or more time periods are pre-configured for each energy graph template based on historical data of the person, wherein the historical data of the person comprises at least one of sleep data, a wakeup time, sensor data on log-in time of the person on the smart desk, and how long the person performed the event at the smart desk, and an age of the person;
a microprocessor that is communicatively connected to the sensor unit and a user device associated with the user, wherein the microprocessor is configured to
receive user data of a particular day from the user device, wherein the user data comprises at least one of sleep data, an age, and a wakeup time of the user on the particular day;
determine an energy graph template for the user for the particular day from the database by analyzing (i) the user data that is received from the user device, and (ii) the log-in time of the user on the smart desk that is received from the sensor unit, for the particular day;
determine an occupancy of the smart desk by the user on the particular day by analyzing information related to the presence of the user on the smart desk, and how long the user is performing the event at the smart desk;
dynamically modify the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user, for the particular day, wherein the adjusted energy graph comprises one or more adjusted time periods on which the potential deceleration in the brain activity of the user occurs;
generate a reminder for each adjusted time period of the adjusted energy graph at a user interface on the smart desk to enable the user to take the break to recover from the deceleration of the brain activity.

2. The smart desk as claimed in claim 1, wherein the sensor unit detects an infra-red radiation surrounding the smart desk to detect the presence of the user on the smart desk.

3. The smart desk as claimed in claim 2, wherein the sensor unit comprises a passive infra-red (PIR) sensor that detects the presence of the user on the smart desk, the log-in time of the user on the smart desk, and how long the user is performing the event at the smart desk.

4. The smart desk as claimed in claim 1, wherein the user device comprises a pre-determined application through which the user data is inputted by the user for the particular day, wherein the pre-determined application of the user device is paired with the smart desk via a network.

5. The smart desk as claimed in claim 4, wherein the network is selected from a group comprising of Bluetooth, Long Range (LoRa), WiFi, Narrow Band-Internet of Things (NB-IoT), GSM/GPRS module, Zigbee WiFi, Radio Frequency (RF), Zigbee or combinations thereof.

6. The smart desk as claimed in claim 1, wherein the data associated with the occupancy of the smart desk by the user comprises a time duration for which the user occupies the smart desk for performing the event and a time duration on which the user takes rest.

7. The smart desk as claimed in claim 1, wherein the user interface comprises a Light Emitting Diode (LED) that visually indicates the deceleration in the brain activity of the user by changing colour.

8. The smart desk as claimed in claim 1, wherein the deceleration of the brain activity of the user comprises a state attained by the user comprising at least one of fatigue, slump, sleepiness, imagination, daydreaming, irritable feeling, stressed, or lack of drive and motivation.

9. A method of identifying a deceleration in a brain activity of a user when the user performs an event at the smart desk and reminding the user to take a break to recover from the deceleration of the brain activity, the method comprising,
providing the smart desk that comprises a sensor unit, and a brain activity identification unit comprising a memory unit, and a microprocessor;
pre-storing a plurality of energy graph templates on a database in the memory unit, wherein each energy graph template comprises one or more time periods at which a person has the deceleration in the brain activity, wherein the one or more time periods are pre-configured for each energy graph template based on historical data of the person, wherein the historical data of the person comprises at least one sleep data, a wakeup time, sensor data on log-in time of the person on the smart desk, and how long the person performed the event at the smart desk, and an age of the person;
detecting, using the sensor unit, a presence of the user on the smart desk, a log-in time of the user at the smart desk, and how long the user is performing the event at the smart desk;
receiving, by the microprocessor, user data of a particular day from a user device associated with the user, wherein the user data comprises at least one of sleep data, age, and a wakeup time of the user for the particular day;
determining, by the microprocessor, an energy graph template for the user for the particular day from the database by analyzing (i) the user data that is received from the user device, and (ii) the log-in time of the user on the smart desk that is received from the sensor unit, for the particular day;
determining, by the microprocessor, an occupancy of the smart desk by the user on the particular day by analyzing information related to the presence of the user on the smart desk, and how long the user is performing the event at the smart desk;
dynamically modifying, by the microprocessor, the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user for the particular day, wherein the adjusted energy graph comprises one or more adjusted time periods on which the potential deceleration in the brain activity of the user occurs;
generating, by the microprocessor, a reminder for each adjusted time period of the adjusted energy graph at a user interface on the smart desk to enable the user to take the break to recover from the deceleration of the brain activity.

10. The method as claimed in claim 9, wherein the method comprising detecting, by the sensor unit, an infra-red radiation surrounding the smart desk to detect the presence of the user on the smart desk.

11. The method as claimed in claim 10, wherein the sensor unit comprises a passive infra-red (PIR) sensor that detects the presence of the user on the smart desk, the log-in time of the user on the smart desk, and how long the user is performing the event at the smart desk.

12. The method as claimed in claim 9, wherein the method comprising inputting, by the user, the user data to the user device for the particular day through a pre-determined application.

13. The method as claimed in claim 12, wherein the method comprising pairing the pre-determined application of the user device with the smart desk via a network.

14. The method as claimed in claim 13, wherein the network is selected from a group comprising of Bluetooth, Long Range (LoRa), WiFi, Narrow Band-Internet of Things (NB-IoT), GSM/GPRS module, Zigbee WiFi, Radio Frequency (RF), Zigbee or combinations thereof.

15. The method as claimed in claim 9, wherein the data associated with the occupancy of the smart desk by the user comprises a time duration for which the user occupies the smart desk for performing the event, and a time duration for which the user takes rest.

16. The method as claimed in claim 10, wherein the method comprising indicating, by the microprocessor, the deceleration of the brain activity of the user by changing colour of the user interface that comprises a Light Emitting Diode (LED).

17. The method as claimed in claim 10, wherein the deceleration of the brain activity of the user comprises a state attained by the user comprising at least one of fatigue, slump, sleepiness, imagination, daydreaming, irritable feeling, stressed, or lack of drive and motivation.

18. One or more non-transitory computer readable storage mediums storing one or more sequences of instructions, which when executed by one or more processors, performs method of identifying a deceleration in a brain activity of a user when the user performs an event at the smart desk and reminding the user to take a break to recover from the deceleration of the brain activity, the method comprising,
providing the smart desk that comprises a sensor unit, and a brain activity identification unit comprising a memory unit, and a microprocessor;
pre-storing a plurality of energy graph templates on a database in the memory unit, wherein each energy graph template comprises one or more time periods at which a person has the deceleration in the brain activity, wherein the one or more time periods are pre-configured for each energy graph template based on historical data of the person, wherein the historical data of the person comprises at least one sleep data, a wakeup time, sensor data on log-in time of the person on the smart desk, and how long the person performed the event at the smart desk, and an age of the person;
detecting, using the sensor unit, a presence of the user on the smart desk, a log-in time of the user at the smart desk, and how long the user is performing the event at the smart desk;
receiving, by the microprocessor, user data of a particular day from a user device associated with the user, wherein the user data comprises at least one of sleep data, age, and a wakeup time of the user for the particular day;
determining, by the microprocessor, an energy graph template for the user for the particular day from the database by analyzing (i) the user data that is received from the user device, and (ii) the log-in time of the user on the smart desk that is received from the sensor unit, for the particular day;
determining, by the microprocessor, an occupancy of the smart desk by the user on the particular day by analyzing information related to the presence of the user on the smart desk, and how long the user is performing the event at the smart desk;
dynamically modifying, by the microprocessor, the energy graph template of the user for the particular day by adjusting the one or more time periods of the energy graph template to obtain an adjusted energy graph, wherein the one or more time periods of the energy graph template are adjusted by analyzing data associated with the occupancy of the smart desk by the user for the particular day, wherein the adjusted energy graph comprises one or more adjusted time periods on which the potential deceleration in the brain activity of the user occurs;
generating, by the microprocessor, a reminder for each adjusted time period of the adjusted energy graph at a user interface on the smart desk to enable the user to take the break to recover from the deceleration of the brain activity.
